(19) United States
(12) Patent Application Publication
(10) Pub. No.: US 2013/0078161 A1
(43) Pub. Date: Mar. 28, 2013

(54) Rapid Heat Transfer Sterilization System For Surgical Instruments And Devices

(71) Applicants: William Bryan Smith, Bloomington, IL (US); Nelson S. Slavik, Niles, MI (US)

(72) Inventors: William Bryan Smith, Bloomington, IL (US); Nelson S. Slavik, Niles, MI (US)

(21) Appl. No.: 13/427,662

(22) Filed: Mar. 22, 2012

Related U.S. Application Data
(63) Continuation of application No. 61/468,623, filed on Mar. 29, 2011.

(51) Int. Cl. A61L 2/04; A61L 2/16

(52) U.S. Cl. CPC A61L 2/04; A61L 2/16
USPC 422/292; 422/307

(57) ABSTRACT

A device and system is disclosed for sterilizing objects, commonly dental, medical, or veterinary instruments, by enhancing rapid hot air diffusion into an instrument container by means of a sliding cover overlaying an opening in the bottom of the instrument container when in the closed position and sliding horizontally to reveal the opening in the open position, allowing rapidly flowing hot air into the container for medical article sterilization. The sliding cover is moved to the open position just prior to the sterilization process by a mechanical means incorporated into the sterilizer with the sliding cover remaining in the open position through the completion of the sterilization process. Once the sterilization cycle is complete, the mechanical means is reversed, closing, sealing, and locking the sliding cover in place over the opening before the instrument container is removed from the sterilizer, ensuring continued medical instrumentation sterility inside the container.

### BACKGROUND OF THE INVENTION

This is a continuation in part of U.S. patent application Ser. No. 61/468,623 filed Mar. 29, 2011.

### FIELD OF INVENTION

The present invention relates to an apparatus and method for sterilization of items. These may be instruments used in medical, dental, veterinary, or other markets, or for the sterilization of other items. There is a need for a device to quickly and efficaciously sterilize objects, such as medical instruments in hospitals, clinics, dental facilities, veterinary, and laboratory facilities.

Currently, steam sterilization is used in hospitals and clinics to sterilize the majority of instruments or other devices. Steam sterilization is also utilized in time of critical instrument need by modifying standard operating protocols and “flashing” steam to the unwrapped instrument or device. The reduced time-temperature profiles of such a technique are often not evaluated for sterilization assurance and have resulted in instruments and devices remaining microbiologically contaminated after treatment. “Flash sterilization” is not a recommended procedure, but in times of critical instrument or device need, it is the only relatively quick method of sterilization available to the medical facility.

U.S. Pat. No. 4,923,681 issued May 8, 1990 to Cox et al. discloses a High Velocity Hot Air Sterilization Device with Controller. High velocity hot air sterilization technology has the potential to meet the sterilization needs of the medical facility as both a standard and expeditious sterilization technology for heat-resistant instruments or devices. However, the original design of the unit limited its usefulness due to its inability to accommodate closed instrument containers that could assure internal conditions for instrument sterilization and yet maintain the sterility of those instruments from environmental microbial contamination once the instrument container was removed from the sterilizer chamber.

The present invention serves to remedy these inadequacies by incorporating described design features into a high velocity hot air sterilization device or other similar devices that are integrated with novel design features of the described instrument container.

These designs allow the introduction of high velocity air into the container under conditions that lead to instrument or device sterilization, yet provide the physical containment necessary to protect sterilized instruments from external environmental microbial contamination once the container has been removed from the sterilizer.

### DESCRIPTION OF PRIOR ART

U.S. Pat. No. 4,923,681 issued May 8, 1990 to Cox et al. discloses a High Velocity Hot Air Sterilization Device with Controller. This device was designed and marketed for use in the dental market to rapidly sterilize small instruments without instrument corrosion. The unit as designed was small and built to accommodate a limited volume of the smaller dental instruments requiring sterilization. With the Cox High Velocity Hot Air Sterilization Device wrapped or unwrapped instruments are placed into a wire mesh, open basket and held for pre-designated times at 375° F. as previously prescribed by the U.S. Food and Drug Administration approval (K8726643A and K881371) for (1) unwrapped instruments, (2) air rotor hand pieces or for instruments with air or water tubing, and (3) wrapped instruments. Upon completion of the sterilization cycle the basket containing the instruments is removed from the sterilizer. Unless covered with a sterile covering, unwrapped instruments are immediately subjected to potential external microbial contamination. For the dental client, this practice is acceptable since sterilization of dental instruments has placed emphasis on obtaining complete kill of microorganisms emanating from previous patients with no concern regarding post-sterilization contamination from microbial contaminants having environmental origins.

For the healthcare and veterinary client, sterilized instruments and devices must retain their sterility prior to their entry into the sterile surgical field and as such, cannot be subject to post-sterilization microbial contamination. In healthcare and veterinary care, protection of sterilized instruments is maintained by wrapping instruments in sterile wrap and subsequently subjecting them to the sterilizing agent (i.e., wet steam heat, dry heat, radiation, or chemical agent) or by placing the instrument(s) in a closed container designed to allow that particular sterilizing agent to migrate through and to have contact with the contained instruments, thus achieving instrument sterilization.

Although wrapping instruments had been a primary mechanism of maintaining instrument sterilization using wet steam heat, static dry heat, radiation, and chemical agents in the past, emphasis has shifted to the use of closed containers for sterilizing larger quantities of instruments and providing subsequent protection from environmental microbial contaminants. With the increased use in healthcare of closed container systems, the use of closed containers in dental clinics has also become the preferred way to protect and store sterilized dental instruments.

Closed containers allowing migration of the sterilizing agent into the container for instrument sterilization have been developed to accommodate specific sterilizing agents. The design of the container and/or its portal design must be congruent with the attributes of the sterilizing agent and not interfere with the influx of the sterilizing agent. Accordingly the design must assure in some manner, the protection of the sterilized instruments from microbial agent contamination from the point of exiting the sterilizer until the container is opened for instrument use.

Closed containers have been designed to incorporate top and bottom perforations protected by a microbial filtering material that is permeable to gas or vapor sterilants, but is impermeable to microorganisms. These perforations may be static, remaining continuously open and filtered. An example of such a container is contained in U.S. Pat. No. 4,551,311 issued Nov. 5, 1985 to Lorenz and entitled “Sterilizer Container.”

Another design incorporates open side vents (U.S. Patent Application Publication No.: US 2003/0211023 Al; Su-Syin Wu and Charles Howlett; “Instrument Sterilization Container Having Improved Diffusion”) to allow gas or vapor sterilants into the container. Protection from microbial contaminants is accomplished through the incorporation of internal or external microbial filters by wrapping the instruments or wrapping the entire container.

The container may also be of a non-static design, providing an automatic opening and shutting mechanism. For steam sterilization the pressure differential between the inside and outside of the container triggers an automatic opening and closing of a pressure-sensitive valve (U.S. Pat. No. 5,352,416 issued Oct. 4, 1994 to Wagner and entitled “Valve Arrangement for a Sterilization Container”).

Rapid heat transfer sterilizers employ rapidly flowing hot air over the surface of an article to affect microbial kill. Hot air influx into the container at a sufficient rate is therefore necessitated to achieve sterilization in the prescribed time-temperature profile previously approved by the U.S. Food and Drug Administration for the Cox unit. Any barrier to that necessitated rate of airflow will significantly impact sterilization conditions. Our research has demonstrated that container perforation coupled with fabric filtration will disturb hot air influx into the container and to the instrument and has significant impact on the conditions necessary to achieve reliable instrument sterilization. Existing instrument containers that employ perforations in the top, sides, and/or bottom of the container also require fabric filtration to mitigate microbial contaminants and thus, prohibit the necessary conditions required for instrument sterilization by high velocity dry heat. Existing instrument containers that utilize pressure valves were specifically designed for pressurized wet steam sterilizers and do not function under the non-pressurized treatment conditions employed in high velocity dry heat sterilization.

The need exists for a high velocity hot air sterilization device integrated with a novel, closed container system that can provide a mechanism to allow for the access of sufficiently flowing high velocity hot air to instruments/devices for their sterilization and also allow for the container to subsequently protect the sterilized instruments from microbial contaminants having environmental origin once the container has been removed from the sterilizer.

### SUMMARY OF THE INVENTION

The present invention provides a rapid transfer dry-heat sterilization system for sterilizing medical, dental, or veterinary instruments or other devices or for other purposes. It is the object of the invention to expand the utilization of the sterilization device as embodied in the invention of Cox et al. More specifically the invention provides: (1) the ability to sterilize trays of instruments and devices within a closed container having the design to allow high velocity dry heat air to enter the container and sterilize contained instruments under the sterilization temperature and time parameters prescribed by the U.S. Food and Drug Administration and (2) the ability to subsequently protect the sterilized instruments from microbial contaminants of ambient environmental origin upon removal of the container from the sterilizer.

Thus, the present invention relates to a covered sterilization container system comprising a lid, sidewalls, and bottom. The lid and sidewalls form an enclosure. The base is comprised of a cut-out portion (1) that is covered by a sliding sealable cover or cap when the container is outside the confines of the high velocity hot air sterilizer, (2) that is uncovered during or subsequent to the container's entry into the sterilizer and during the sterilization cycle, and (3) that is covered and sealed during or prior to the exit of the container from the sterilizer. The present invention also provides integrated sterilizer-container mechanisms to move the sliding cover or cap from closed to open to closed positions during or subsequent to the entry into and during or prior to exit from the sterilizer. Furthermore, the invention also provides locking and unlocking mechanisms for the sliding cover or cap to ensure its placement across the open portion of the container base so that the instruments and devices in the container maintain sterility after the sterilization process. In addition, the invention provides mechanisms for a tight seal of the sliding cover or cap against the container base when the sliding cover or cap is in the closed position. Additionally, the invention provides for (1) a lid of the container to allow hot air diffusion out of the container, but microbiologically filtered to allow for enhanced cooling of the instruments after the sterilization process and (2) a lid having internal baffling to enhance hot air circulation throughout the container during the sterilization cycle.

Preferably, the container and all its subparts are comprised of materials able to withstand the rigors presented by the temperatures utilized in high velocity hot air sterilization (375 degrees F. or higher). Preferably, these materials include primarily stainless steel, high temperature resistant thermoplastic and thermosetting polymers, ceramics, silicone, and nylon fabric plastics.

Preferably, the container is positioned into the sterilizer by its placement onto a sliding tray, which guides the container into and out of the sterilizer and assures the open-close mechanisms for the sliding cover or cap are properly aligned and positioned.

Preferably, the container base is constructed with an open bottom portion to allow the high velocity hot air into the container from the sterilizer when the sliding cover or cap is in the open position.

Preferably, over the open portion of the container is a series of baffles that direct the high velocity air from the sterilizer to all parts of the container.

Preferably, the open portion of the container bottom is uncovered by means of a sliding cover or cap which is pushed or pulled away from the opening during or subsequent to the container's entry into the sterilizer and which is re-covered by the sliding cover or cap when pushed or pulled back to the closed position prior to or during the removal of the container from the sterilizer. The sliding cover or cap may be mounted either internally or externally to the bottom of the instrument container.

Preferably, the sliding cover or cap is moved to the open or closed position by the use of a fixed or movable mechanism attached to the sterilizer base and which engages with the sliding cover or cap on a instrument container or sliding covers or caps on multiple instrument containers on single or multiple levels having one or more air plenums within the sterilizer.

Preferably, a sliding cover or cap is guided by means of two parallel rails allowing the sliding cover or cap to transverse from the closed position at entry or subsequent to entry into the sterilizer to the fully open position during the sterilization cycle, and back to the closed position prior to or upon the container's exit from the sterilizer. Stops at either end of the rails ensure the sliding cover or cap remains between the rails and assure the correct positioning of the sliding cover or cap. Depending on the mounting location of the sliding cover or cap, these rails may be attached internally or externally to the bottom of the instrument container.

Preferably, the sliding cover or cap is constructed from a durable heat-resistant metal, plastic, or ceramic material having a silicone or other heat resistant gasket at the container-cover/cap interface to seal the sliding cover or cap against the surface of the base. Preferably, to further assure a tight seal over the opening of the container base, the internal configuration of each rail is sloped so as to tighten the gasket against base when the sliding cover or cap is in the closed and locked position.

Preferably, to further assure that the sliding cover and cap remains in the closed position once the sterilizer container is removed from the sterilizer, a locking mechanism or holding mechanism is engaged.

Preferably, a sliding cover locking or holding mechanism is automatically disengaged during the entry or subsequent to the entry of the container into the sterilizer to allow the sliding cover or cap to move into the open position prior to the sterilization process and is re-engaged once the sliding cover or cap is re-located into the closed position before the container is removed from the sterilizer.

Preferably the sterilizer creates a laminar airflow curtain at the entrance to the sterilizer chamber in a sufficient volume and speed of airflow to preclude entry of microbial contaminants external to the sterilizer during and subsequent to the sterilization process when instrument containers are being withdrawn from the sterilizer chamber.

Preferably, the container has a lid opening that is centered and onto which is placed a spunbond or nonwoven nylon microbial filter sealed in a rigid housing and sealed against the lid to allow heat to escape and instruments to cool after the sterilization process.

Preferably, the container has a lid that contains a baffled internal surface to enhance hot air circulation throughout the container during the sterilization cycle.

### BRIEF DESCRIPTION OF THE DRAWINGS

In describing the invention, reference will be made to the accompanying drawings in which:

FIGS. 1A, 1B, and 1C are perspective views of the instrument container and inverted lid;

FIGS. 2A and 2B are external views of the top of the instrument container lid having a microbial filter and bottom of the instrument container base having an external slide cap in an open position;

FIGS. 3A and 3B are cross-sectional side elevation views of the instrument container with external slide cap and slide rails situated in both closed and open slide cap positions within the high velocity hot air sterilization device;

FIGS. 4A, 4B, and 4C are cross-sectional views of the external slider rail, slide cap, and gasket seal in both open and closed positions;

FIGS. 5A and 5B are cross-sectional views of the external slide cap locking mechanism;

FIGS. 6A, 6B, 6C, and 6D are internal and external views of the bottom of the instrument container exhibiting an internal sliding cover in both open and closed positions;

FIGS. 7A and 7B are cross-sectional side elevation views of the instrument container with internal sliding cover and slide rails situated in both closed and open sliding cover positions within the high velocity hot air sterilization device; and

FIGS. 8A and 8B are isometric and front elevation views of a two-container, internal sliding cover configuration within a sterilizer depicting the push-pull engagement bar with the containers' push-pull bars.

### DETAILED DESCRIPTION OF THE PREFERRED EMBODIMENTS

The present invention provides a sterilization system that utilizes high velocity dry air heat to sterilize medical, veterinary, and dental instruments and devices that are situated in a novel container which allows the high velocity hot air to penetrate and diffuse within that container and which allows such sterilized instruments and devices to remain protected from environmental contaminants upon removal of the container from the sterilizer.

The present invention embodies two distinct mechanisms that can be employed to open and close instrument containers. One embodiment describes a mechanism in which the sterilizer has a fixed engagement bar that physically engages a sliding cap on the container, pushing it to reveal an opening in the container's base during container entry and pulling it back across the opening during the container's exit from the sterilizer. Another embodiment is described in which a pulley push-pull mechanism operated by the door latching and locking mechanism pulls the sliding cover to reveal an opening in the container's base and pushes the sliding cover back across the opening to a closed position once the instrument container has been loaded into the sterilizer.

The present invention is also envisioned applicable for other sterilization systems that may employ steam or sterilant gas or vapor, or heat to inactivate or kill microorganisms by providing direct access of the sterilizing agent to a medical instrument or article during a sterilization process and by protecting the sterilized instruments or articles from environmental contaminants once the container is removed from the sterilizer.

The present invention may also be envisioned to bring air into the instrument container through the top (lid) or sides of the container.

The preferred and described embodiment of the present invention is described below.

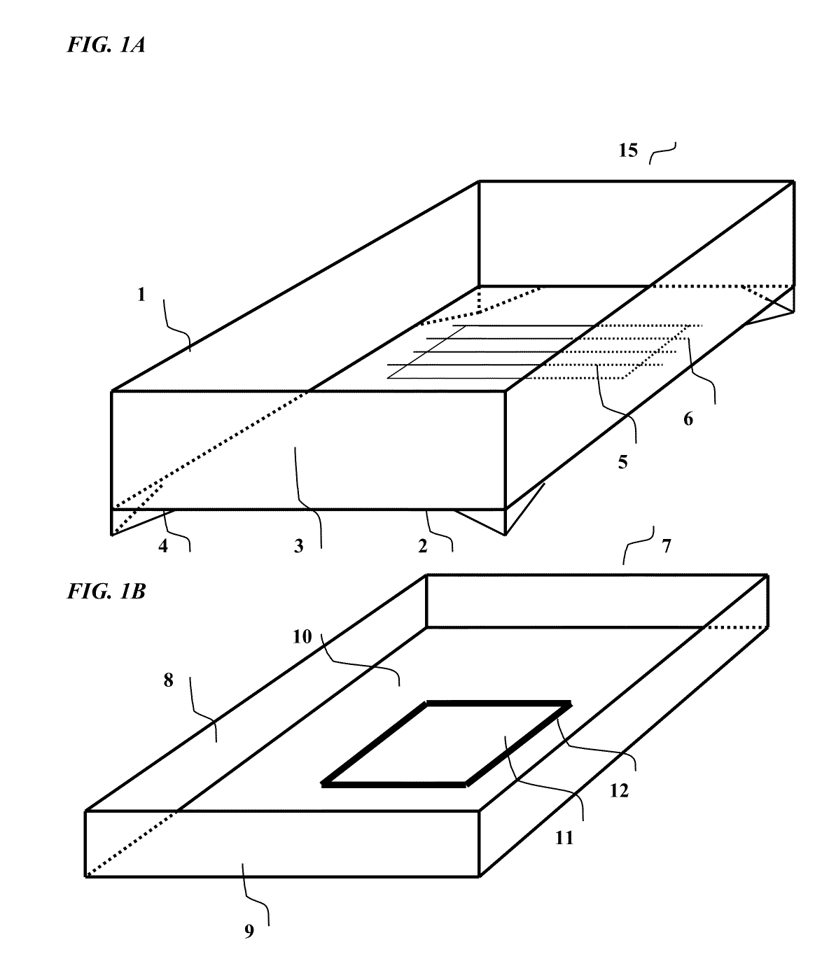

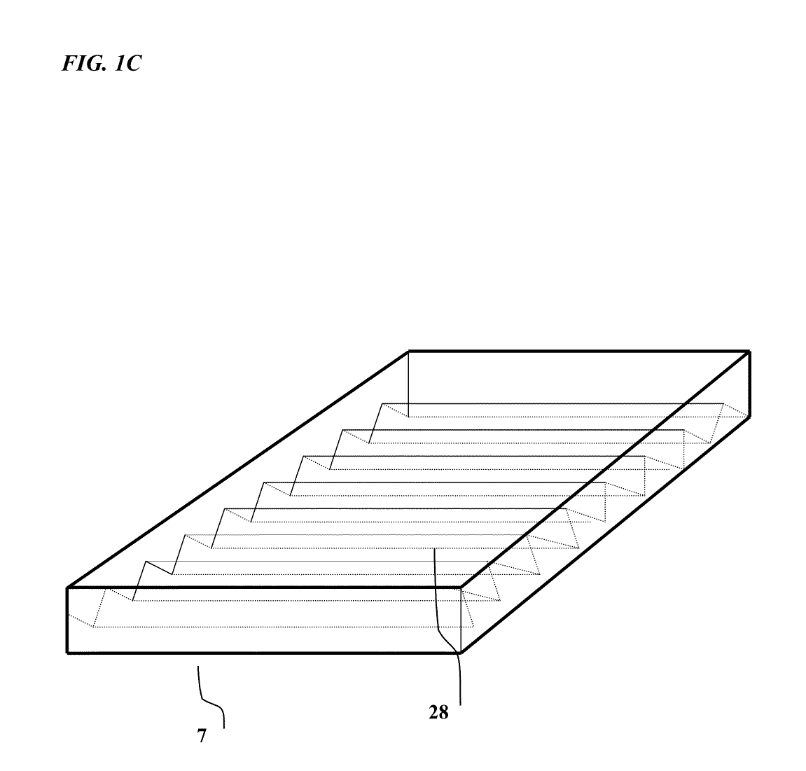

FIG. 1A is a perspective view of the preferred rectangular sterilization container 15. The sterilization container 15 is typically rectangular in shape with solid sidewalls 1 and end walls 2 and having a base 3 on which reside support feet 4 or perpendicular base extensions on its exterior. The solid base 3 contains an air entry portal 5 over which are baffles 6 that serve to deflect forced high-velocity hot air from the sterilizer into all portions of the container 15.

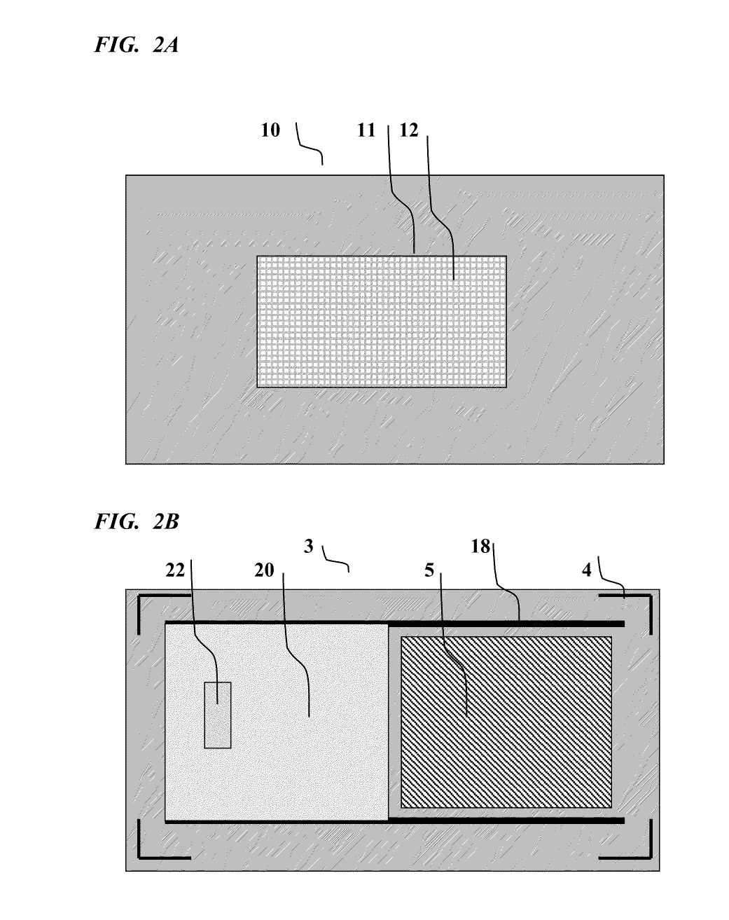

FIGS. 1B and 2A are views of the container lid 7. The lid 7 (shown inverted) has solid sidewalls 8 and end walls 9. FIGS. 1B and 2A depict the container lid top 10, which is solid with a cut out portion 11 containing a nested and sealed thermal-resistant spunbond or nonwoven nylon, removable microbial filter 12 which is designed to assist in the cooling of the interior of the container 15 after the sterilization process. During sterilization the lid 7 is affixed to the container 15 and remains affixed until such time the sterile instruments are required.

FIG. 1C is a perspective view (shown inverted) of the container lid 7 containing a solid baffle 28 to deflect hot air entering the container 15 from the air entry portal 5 during the sterilization process to aid in its diffusion throughout the container 15.

FIGS. 2B, 3, 4, and 5 depict an embodiment in which an instrument container 15 has an external slider cap 20 to which is inserted a fixed engagement bar 31 that is attached to the sterilizer 40. Entry of the instrument container 15 into the sterilizer 40 results in the external slider cap 20 remaining stationary while the container 15 is manually pushed to the rear of the sterilizer 40, thus uncovering the air entry portal 5 and allowing rapidly flowing hot air to enter the container 15 and sterilize instruments. Upon completion of the sterilization process the container 15 is manually pulled to the front of the sterilizer 15, closing and sealing the external slider cap 20 across the air entry portal 5 to preclude microorganisms from contaminating the sterilized instruments. The closed instrument container 15 can now be removed from the sterilizer 40 by lifting up the instrument container 15, thus disengaging the fixed engagement bar 31 from the slider cap's 20 insert well 22.

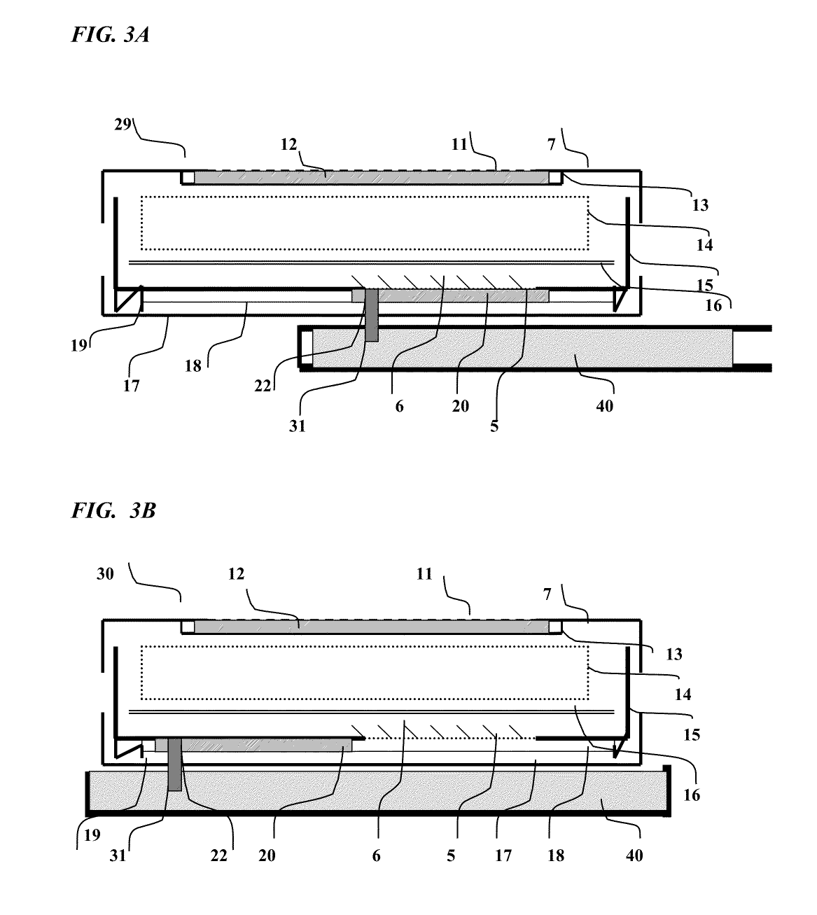

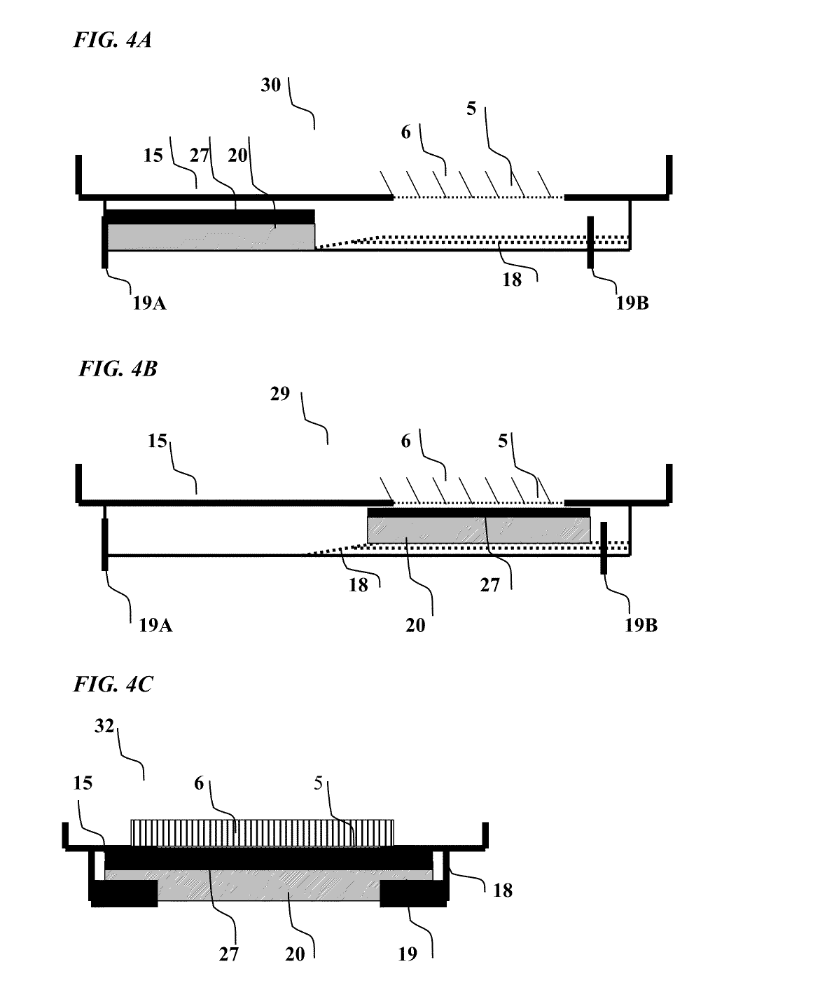

FIG. 2B is a view of the underside of the container base 3 having parallel externally-mounted slide rails 18 along the length of the container base 3 on which rides a slider cap 20 containing an external insert well 22 for the slider cap engagement bar 31 (FIGS. 3A and 3B). The slider cap 20 covers the air entry portal 5 when in the closed position (FIG. 3A and 4B). The underside of the base 3 contains four container support feet 4 that protect the slider cap 20 and parallel mounted slide rails 18 that run along the length of the container base 3.

FIG. 3 depicts a cross-sectional side elevation view of a preferred embodiment of the instrument container 15 and lid 7 with slider cap 20 and slide rails 18 with rail end caps 19 situated in both closed 29 and open 30 slider cap positions within the high-velocity hot air sterilization device 40. The instrument container 15 holds a basket 14 or alternative instrument holding device which contains the instruments to be sterilized. The basket 14 sits on a perforated stainless steel floor covering 16, which provides support for the basket 14 over the opening 5 in the container floor and the air baffles 6. The container 15 is placed into the sterilizer 40 by means of a holding tray 17, which provides guidance and positioning of the container 15 as it is pushed into the sterilizer 40. Positioning and guidance are required to properly seat the slider cap engagement bar 31 into the bar insert well 22 of the slider cap 20. Upon pushing the container 15 into the sterilizer chamber the sliding cap 20 is retained at the front of the sterilizer 40 while the container 15 is pushed to the rear of the sterilizer 40 thus allowing the slider cap 20 to slide away from the air entry portal 5 along the parallel slide rails 18 and allowing high velocity hot air to enter the container 15 from the bottom of the sterilizer 40. Upon completion of the sterilization process the instrument container 15 is pulled from the sterilizer 40 and the process is reversed, sliding the slider cap 20 to cover the air entry portal 5. The sterilized container 15 is removed from the sterilizer 40 disengaging the coupling between the slider cap 20 and slider cap engagement bar 31. Subsequent to the sterilization process when the sterilizer door 41 is opened and instrument containers 15 are withdrawn from the sterilizer 40, the sterilizer 40 continues to generate a laminar airflow curtain at the entrance to the sterilizer 40 chamber that is sufficient in airflow volume and speed to preclude entry into the sterilizer of environmental microbial contaminants. Therefore the laminar airflow curtain also precludes potential microbial entry into the instrument container 15 as the slider cap 20 is pulled to the closed position during the container's 15 removal. At any time during container 15 removal, the leading open edge of the air entry portal 5 always remains near the middle of the sterilizer 40 chamber, thus assuring no microbial contaminants from the outside environment can enter the instrument container 15 during its removal. To assist in the release of hot air retained inside the instrument container 15 after the sterilization process is complete, a removable spunbond or nonwoven nylon microbial filter 12 is contained in the lid 7, sealed in a rigid housing 13 over the opening 11.

FIGS. 4A and 4B depict cross-sectional views of the slider rail 18 and slider cap 20 with attached gasket 27 in both open 30 and closed 29 positions of the air entry portal 5 and air baffle 6 in the instrument container 15. In the open position 30 the slider cap 20 with attached gasket 27 rides loosely on the slider rail 18 with little or no contact to the gasket 27 against bottom of the instrument container 15. In this position the slider cap rests near or touching the front rail end cap 19A. In the closed position 29 the slider cap 20 is pulled back into its position immediately under the air entry portal 5. The slider cap 20 with attached gasket 27 is wedged tightly against the external bottom of the container base 3 immediately surrounding the perimeter of the air entry portal 5 by providing a slope and plateau on the back half of the interior bottoms of the parallel slide rails 18 causing the slider cap 20 to rise slightly and compress the attached gasket 27 tightly against the external bottom of the container base 3. The rear rail end caps 19B prevent the slider cap 20 from going beyond the correct sealable position. A rear view 32 is depicted in FIG. 4C of the slider rail 18, slider cap 20 with attached gasket 27, and rail end caps 19 in the closed 29 position under the air entry portal 5 and air baffle 6 in the instrument container 15. The gasket 27 preferably is composed of a high-heat resistant material such as silicone or other comparable material that can be successfully compressed against the bottom of the container base 3. The slider cap 20, slide rails 18, and rail end caps 19 are preferably composed of a hard, high-heat resistant plastic material or other material as found suitable for the application.

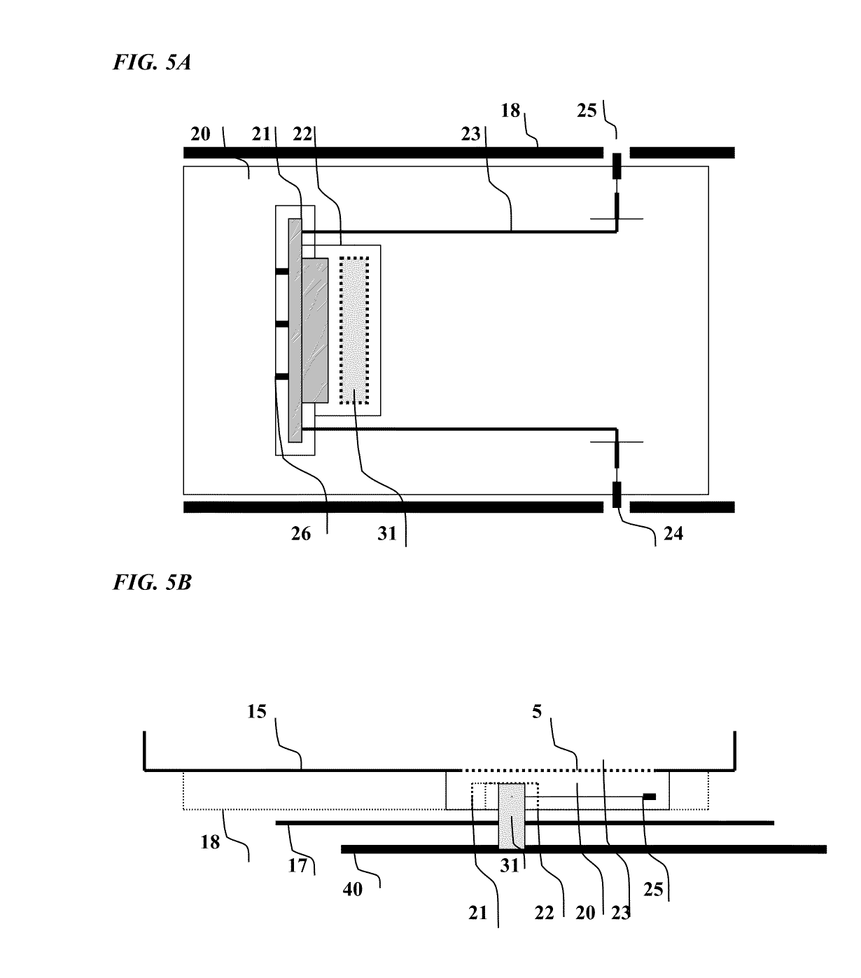

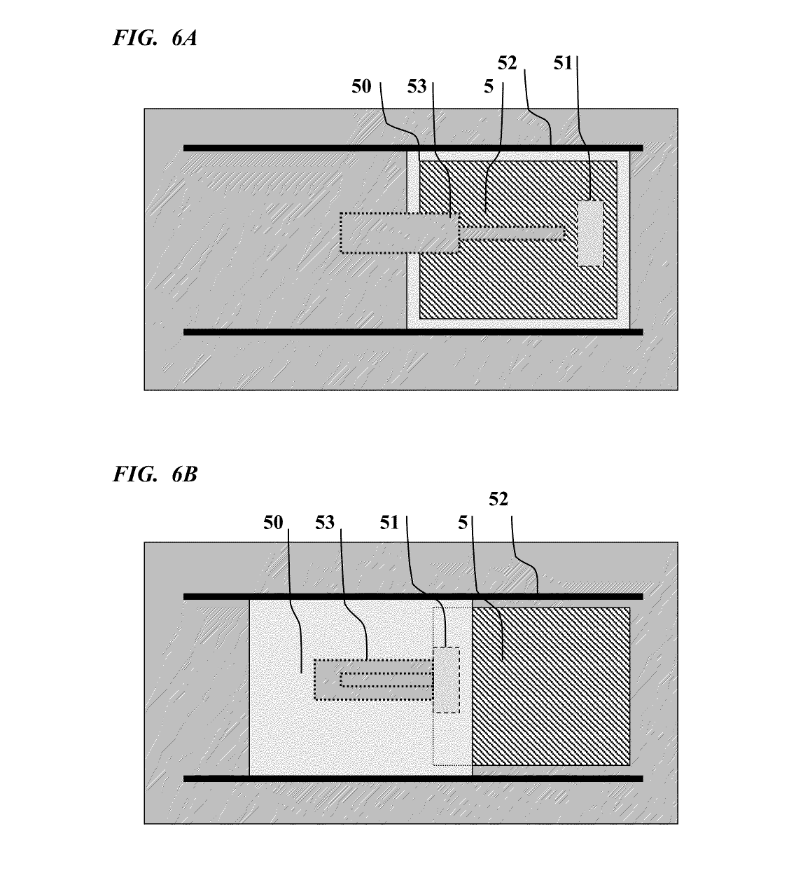

FIGS. 5A and 5B depict a cross-sectional top and side elevation views, respectively, of a preferred embodiment of a slider cap locking mechanism to retain the slider cap 20 in its closed position. Channeled internally into the slider cap 20 are two cables 23 attached to a spring 26 -loaded push bar 21 on one end and on the other end to two spring-loaded pins 24 which are actively engaged into holding slots 25 located internally on each slide rail 18 at the back end of the slide rail 18 and in a position to lock the slider cap 20 in place directly centered beneath the air entry portal 5 in the instrument container 15. Upon entering the sterilizer 40, the instrument container 15 is lowered onto the holding tray 17 and positioned by inserting the slider cap engagement bar 31 into the bar insert well 22 of the slider cap 20. By pushing the instrument container 15 toward the back of the sterilizer 40 the slider cap 20 is retained in place, pushing the slider cap engagement bar 31 against the spring 26-loaded push bar 21 toward the front of the bar insert well 22 thus tightening the two cables 23 attached the two spring-loaded pins 24 and pulling them out of their respective holding slots 25 in the slide rails 18. The slider cap 20 is then free to move to the front portion of the container 15 in its open position. At the completion of the sterilization cycle, the instrument container 15 is pulled out of the sterilizer 40, thereby forcing the slide cap engagement bar 31 against the back portion of bar insert well 22 and releasing the spring-loaded 26 push bar 21 to relax the two cables 23 and cause the two spring-loaded pins 24 to re-engage into their respective holding slots 25, locking the slide cap 20 back in its closed position. Although the preferred embodiment is the use of spring-loaded pins and push bar, it is envisioned that other locking mechanisms may also serve to lock and unlock the slider cap 20 which may or may not include springs or cables and may include the use of dense memory foams or metal clips.

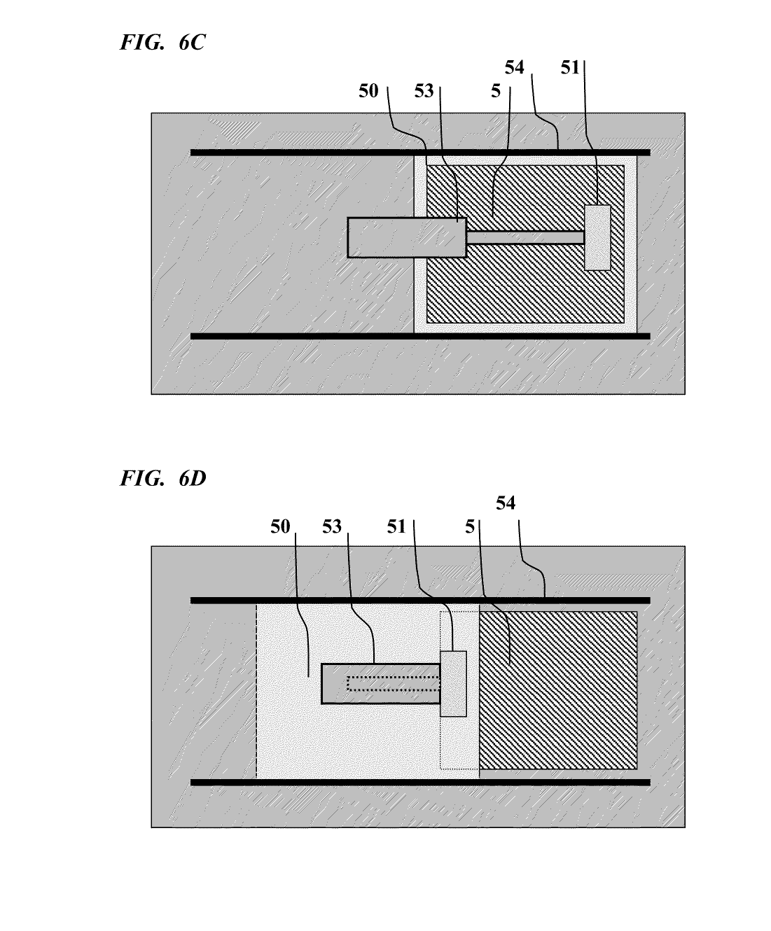

FIGS. 6, 7, and 8 depict a second embodiment in which an instrument container 15 has an internal sliding cover 50 which is moved from a closed position over the air entry portal 5 by a pulley 44 pull-push mechanism connected to the sterilizer door 41 latching and locking apparatus. Upon positioning the container 15 on the holding tray 17 and insertion into the sterilizer 40, a cable 45 is pulled forward via a pulley 44 when the door handle 42 is turned to its closed and locked position, pulling an internal sliding cover 50 and opening the air entry portal 5. The mechanism is reversed upon completion of the sterilization cycle when the sterilizer door 41 is unlocked and the latch is positioned to the open position, pushing the cable 45 and thus the sliding cover 50 into the closed position over the air entry portal 5. The closing of the sliding cover 50 is aided by a spring-loaded piston 53 or similar device located on the external bottom of the container base 3. The spring-loaded piston 53 also serves to lock or hold the sliding cover 50 in place over the air entry portal 5.

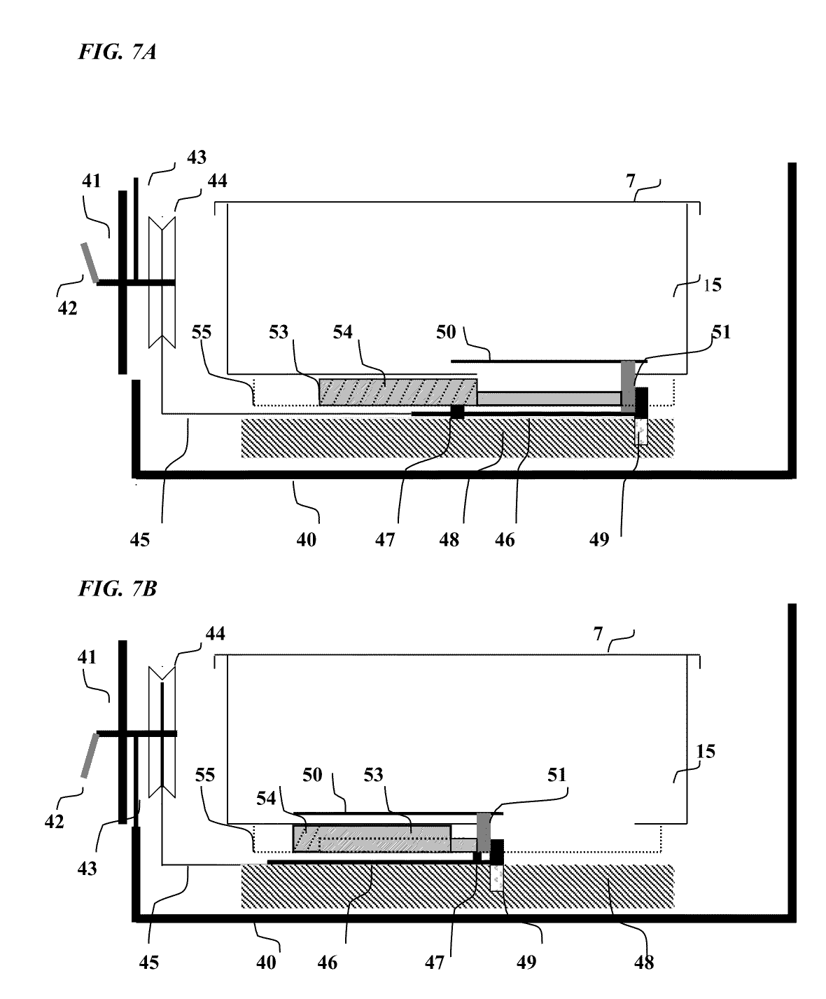

FIGS. 6A and 6B and FIG. 6C and D depict the internal sliding cover 50 configuration on the inside and outside, respectively, of the container base 3 in both the closed (FIGS. 6A and C) and open positions (FIGS. 6B and D). Two parallel sliding cover rails 52 position the sliding cover 50 within the container 15 and allow it to slide freely from the closed position covering the air entry portal 5 to the open position uncovering the air entry portal 5. Attached to the bottom of the sliding cover 50 is a sliding cover push-pull bar 51 extending downward from the sliding cover 50 to engage a push-pull engagement bar 49 that pushes the sliding cover 50 to the open position uncovering the air entry portal 5 when the sterilizer door 41 is latched and locked (FIG. 7). Parallel external rail guides 55 located externally on the container base 3 assist in the positioning of the push-pull engagement bar 49 during the uncovering and covering of the air entry portal 5. A push-pull compression spring piston 53 is used to assist in the return of sliding cover 50 to the closed position covering the air entry portal 5 and to hold the sliding cover 50 in place to prevent microbial contamination of sterilized instruments during storage. The compression end of a push-pull spring piston 53 is externally attached to the instrument container base 3 and the distal piston end is attached to sliding cover push-pull bar 51. The push-pull spring piston 53 is in the extended mode when the sliding cover 50 is covers the air entry portal 5 (FIG. 6A and 6C) and is compressed when the sliding cover 50 has been pushed to the open position (FIG. 6B and 6D), uncovering the air entry portal 5 to allow hot rapidly flowing air entry into the instrument container 15. Although the preferred embodiment is the use of a push-pull compression spring or rod, or combination thereof, it is envisioned that other similarly functional devices or mechanisms may also be employed. Alternative means to move the sliding cover 50 or slider cap 20 are also envisioned using other mechanical means with (e.g., electromagnetic means or servo motors) with or without electrical or hydraulic/pneumatic assist and with or without pulley assist (e.g., levers or other mechanical means).

FIG. 7 depicts a cross-sectional side elevation view of a preferred embodiment of the instrument container 15 incorporating an internal sliding cover 50. FIG. 7A depicts the sliding cover 50 in the closed position over the air entry portal 5 and FIG. 7B depicts the sliding cover 50 in the open position aside the air entry portal 5. The sliding mechanism is activated once the container 15 has been placed on the holding tray 17, inserted completely into the sterilizer 40, and during the closing and locking the sterilizer door 41. Rotating the sterilizer door latch handle 42 engages the sterilizer door latch 43 to the locking position, simultaneously rotating the cable pulley 44 to which is attached a cable 45. Pulled around the cable pulley 44, the cable 45 pulls the push-pull engagement bar 49 forward by way of a push-pull rod 46 intermediate. Thus engaged, the sliding cover push-pull bar 51 pushes the sliding cover 50, positioned by the parallel external guide rails 55, to uncover the air entry portal 5 to the backstop 47. The push-pull engagement bar 49 is held in position and kept on track by way of push-pull engagement bar guide 48.

As the push-pull engagement bar 49 pushes the sliding cover push-pull bar 51, the push-pull spring piston 53 compresses against the internal (or external) compression spring 54. The push-pull spring piston 53 is held in this position until the sterilizer door latch 43 is released and the sterilizer door latch handle 42 is counter-rotated causing the cable pulley 44 to unwind the cable 45 and release the tension to the push-pull engagement bar 49. As the tension is released, the pressure against the push-pull spring piston 53 is subsequently lessened and the push-pull spring piston 53 pushes the sliding cover push-pull bar 51 and sliding cover 50 back to the closed position where the remaining tension in the push-pull spring piston 53 holds the sliding cover 50 in place over the air entry portal 5.

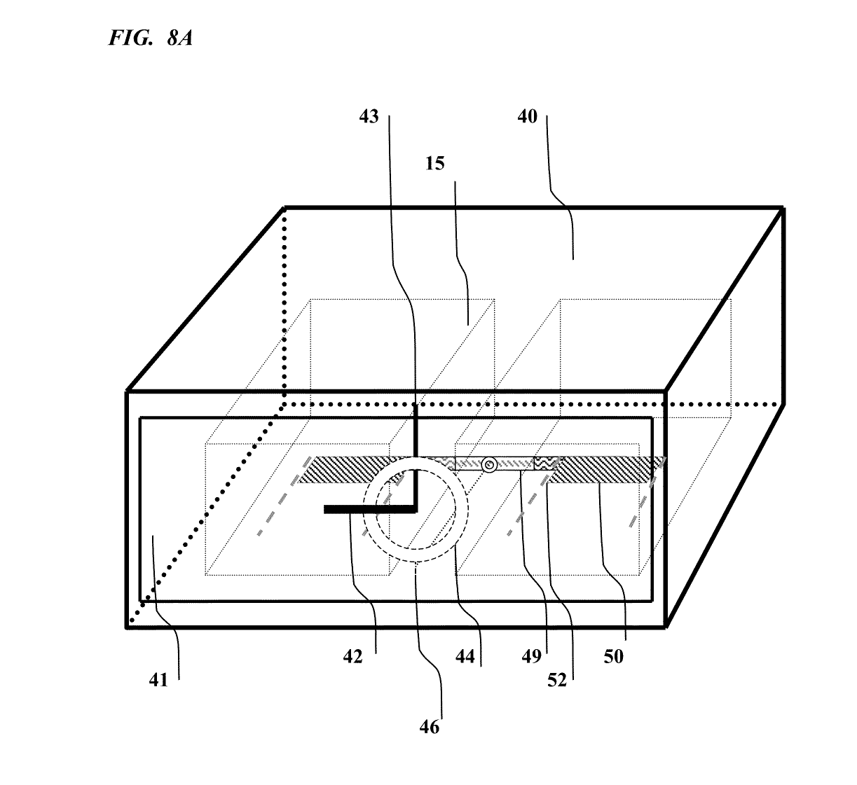

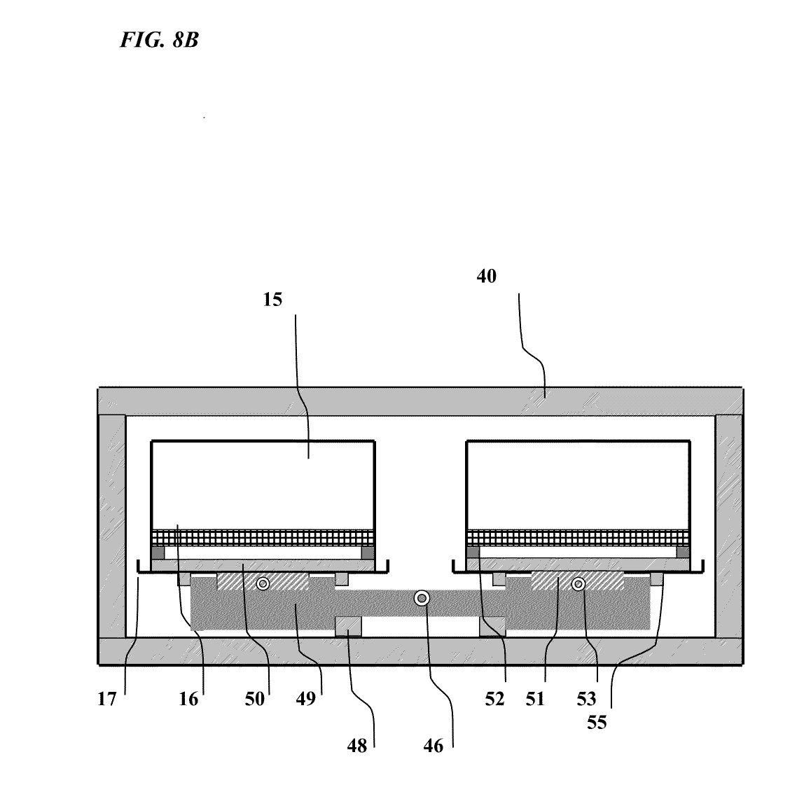

FIG. 8A and FIG. 8B represent the spatial configuration of the push-pull engagement bar 49 within the sterilizer 40 in relationship to an instrument container or containers 15, holding tray 17, and the sliding cover push-pull bar 51. FIG. 8 depicts a sterilizer 40 holding two instrument containers 15 side-by-side with the push-pull engagement bar 49 engaging both containers 15 and pulled/pushed by a single cable 46 attached to the cable pulley 44. The push-pull engagement bar 49 is positioned and guided by parallel push-pull engagement bar guides 48 attached in the sterilizer 40 floor. This configuration allows multiple containers 15 to be handled by a single push-pull engagement bar 49 whether on a single plane as depicted and/or on multiple tiers or levels.

What is claimed is:

1. a sterilization means having the ability to sterilize medical and research articles and the like with a sterilant whether a static dry heat, flowing dry heat, wet heat, a chemical agent or the like acting as sterilant or a microbiological inactivation agent, comprising:
an instrument container having an unfiltered opening or openings, therein, said container being configured to be placed within said sterilizer means for the purpose of instrument or medical article sterilization;
a container opening cover located across the unfiltered opening of the said instrument container and being movable away from the instrument container opening to allow an exchange of the steriliant through said opening from the sterilizer to the interior of the instrument container during a sterilization cycle and being returned to a closed, locked and sealed position across the opening upon completion of the sterilization cycle;
a sterilizer means having a mechanism to engage the container opening cover wherein the container opening cover is moved within the sterilizer during the instrument container's insertion into the sterilizer or prior to the initiation of sterilization cycle to allow the container opening to be unobstructed for sterilant exchange between the sterilizer and the interior of the instrument container;
a sterilizer means having a mechanism to engage the container opening cover wherein the container opening cover is closed, sealed and locked within the confines of the sterilizer subsequent to the completion of the sterilization cycle;
a container opening cover means having a mechanism to accept a sterilizer engagement mechanism to move the container opening cover to aside the instrument container opening to allow an unobstructed opening for sterilant exchange between the sterilizer and the interior of the instrument container; and
a container opening cover means having a configuration to accept a sterilizer engagement mechanism to move the container opening cover across the instrument container opening to allow the container opening cover to overlay the opening and seal and lock the container opening cover in place to preclude entry of environmental contaminants including microorganisms after the sterilization process.

2. A sterilization system according to claim 1, wherein a sliding tray mechanism has a configuration to load, position, and guide the instrument container within the sterilizer chamber to ensure proper engagement between the container opening cover and a sterilizer engagement mechanism for uncovering and covering the container opening.

3. A sterilization system according to claim 1, having a sterilizer that has a fixed engagement mechanism interacting with the container opening cover of the instrument container that allows the positioning and re-positioning of the container opening cover during manual inserting and removal of the instrument container.

4. A sterilization system according to claim 1, having a sterilizer that has a movable engagement mechanism interacting with the container opening cover of the instrument container that allows positioning and re-positioning of the container opening cover during the process of latching/unlatching and locking/unlocking the sterilizer door or by other means.

5. The apparatus of claim 4, having a movable engagement mechanism operated mechanically by pulleys, cables, and push-pull compression spring devices or with electromagnetic means or servo motors to allow positioning and re-positioning of the container opening cover.

6. The apparatus of claim 4, having a sterilizer that has parallel guide rails affixed to the exterior base of the sterilizer to guide and to position a movable sterilizer engagement mechanism for uncovering and covering the instrument container opening.

7. A sterilization system according to claim 1, wherein the sterilizer has a fixed or movable engagement mechanism that has the capacity to engage one or more container opening covers on one or more instrument containers on one or more levels of containers within a sterilizer.

8. A sterilization system according to claim 1, wherein the sterilizer creates a laminar airflow curtain at the entrance to the sterilizer chamber in a sufficient volume and speed of airflow to preclude entry of microbial contaminants external to the sterilizer during and subsequent to the sterilization process when instrument containers are being withdrawn from the sterilizer chamber.

9. A sterilization system according to claim 1, wherein the instrument container has a sealable lid containing an air baffling configuration to enhance sterilant circulation throughout the interior of the instrument container.

10. A sterilization system according to claim 1, wherein the instrument container has a series of directional airflow baffles attached to the interior of the container and across the container opening.

11. A sterilization system according to claim 1, wherein the instrument container has perforated or wire mesh container floor covering to protect a container base opening, directional airflow baffles, and container opening cover.

12. A sterilization system according to claim 1, wherein the sterilization container has protruding support feet or perpendicular extensions to support the container and protect any extended container opening cover and guide mechanisms that may protrude from the bottom of the instrument container.

13. A sterilization system according to claim 1, wherein the instrument container has parallel guide rails to guide an opening cover from its position across the instrument container opening to a position away from the instrument container opening and reverse.

14. A sterilization system according to claim 1, wherein the instrument container has parallel guide rails affixed to the exterior base of the container to guide and position a movable sterilizer engagement mechanism for uncovering and covering the instrument container opening.

15. A sterilization system according to claim 1, wherein the instrument container and container opening cover have a configuration to ensure a tight seal when the container opening cover is positioned across the instrument container opening and loose fitting when the container opening cover is positioned away from the instrument container opening.

16. A sterilization system according to claim 1, wherein the container opening cover has a mechanism to assure the container opening cover is locked or remains in place when in the covered position across the instrument container opening and is released during the process of uncovering the instrument container opening.
